(19) United States
(12) Patent Application Publication
(10) Pub. No.: US 2008/0125456 A1
(43) Pub. Date: May 29, 2008

(54) Injection of Tacrolimus

(75) Inventors: Sang Joon Lee, Gunpo-si (KR); Hee Jong Shin, Bucheon-si (KR); Seok Kyu Lee, Cheonan-si (KR); Ji Hun Yun, Cheonan-si (KR)

Correspondence Address: SUGHRUE MION, PLLC, 2100 PENNSYLVANIA AVENUE, N.W., SUITE 800, WASHINGTON, DC, 20037, US

(73) Assignee: Chong Kun Dang Pharmaceutical Corporation, Seoul (KR)

(21) Appl. No.: 11/718,547

(22) PCT Filed: Nov. 11, 2005
(86) PCT No.: PCT/KR05/03814

§ 371 Date: May 3, 2007

(30) Foreign Application Priority Data
Nov. 12, 2004 (KR) 10-2004-0092274

(51) Int. Cl. A61K 31/436

(52) U.S. Cl. 514/294

(57) ABSTRACT

This invention relates to tacrolimus injection comprising tacrolimus as an active ingredient, macrogol 15 hydroxystearate as a surfactant and a non-aqueous solvent.

### TECHNICAL FIELD

This invention relates to tacrolimus injection comprising tacrolimus as an active ingredient, macrogol 15 hydroxystearate as a surfactant and a non-aqueous solvent.

### BACKGROUND ART

Tacrolimus in this invention is the INN name of the compound 17-allyl-1,14-dihydroxy-12-[2-(4-hydroxy-3-methoxycyclohexyl)-1-methylvinyl]-23,2 5-dimethoxy-13,19,21,27-tetramethyl-11,28-dioxy-4-azatricyclo[22.3. 1.0.^{4.9}]octacos-1 8-ene-2,3,10,16-tetraone (Hereinafter referred to as “tacrolimus”).

The tacrolimus which has immunosuppressive and antimicrobial activities is useful for treatment and/or prevention of the following diseases and conditions:

Rejection reactions by transplantation of organs or tissues;

Graft-versus-host reactions following bone marrow transplantation;

Autoimmune diseases, and

Infections caused by pathogenic microorganisms.

Tacrolimus is well dissolved in an organic solvent (for example, methanol, ethanol or acetone) and is practically insoluble in water.

The conventional tacrolimus formulations have been chiefly prepared in a solid form such as capsule, but there are many approaches to improve dissolution rate because of insolubility of the drug, via grind of drug particles, enhancement of drug solubility not only by the addition of a surfactant, but also by the manufacture of microemulsion or solid dispersion formulations.

The Japan Patent Laid-open No. Sho 62-277321 disclosed a solid dispersion composition comprising tacrolimus and a water-soluble polymer as a carrier.

The U.S. Pat. No. 6,346,537 disclosed a pharmaceutical composition comprising tacrolimus, surfactant(s), and solid carrier(s) such as water-soluble polymers, saccharides, and light anhydrous silicic acid. The composition in this patent is a solid dispersion that both tacrolimus and surfactant(s) are dispersed into the solid carrier(s), followed by removal of an organic solvent.

Despite a variety of approaches, many pharmaceutical formulations of tacrolimus cannot be effective in oral administration because of the poor bioavailability. It is due to low dissolution rate of the drug and/or low absorption rate from the gastrointestinal tract. Thus unable to reach desired blood concentration within a short period of time.

Furthermore, the oral formulation is not easily administered to children or weak or unconscious patients.

There is a need, therefore, for tacrolimus formulations that can deliver the active ingredient intravenously to rapid efficacy, have a high bioavailability, and available to patients who cannot receive oral administration.

The Korean Patent No. 0177158 disclosed a solution formulation comprising tacrolimus or its pharmaceutically acceptable salt, pharmaceutically acceptable surfactant including polyoxyethylene hydrogenated castor oil, and pharmaceutically acceptable non-aqueous solvent,

The Korean Patent No. 0206722 disclosed a solution composition comprising tacrolimus or its pharmaceutically acceptable salt, a pharmaceutically acceptable emulsifier selected from the group comprising egg york lecithin, soybean lecithin, polyoxyethylene hydrogenated castor oil, and a pharmaceutically acceptable oil from the liquid hydrocarbon group comprising soybean oil and sesame oil.

The Korean Patent Unexamined Publication No. 2001-0006070 disclosed a pharmaceutical composition comprising a water-insoluble drug and two or more of surfactants, characterized in that at least one surfactant dissolves both other water-insoluble surfactant(s) and water-insoluble drug.

However, the solution compositions of the prior art have some problems that the drug tends to be precipitated during long-term storage, the pharmaceutical stability may go down with decrease of drug content. Thus, some of the conventional solution compositions are not suitable for human as an injectable formulation.

Surfactant is a wetting agent that reduces surface tension in solution. It has a linear molecule with a hydrophilic head and a hydrophobic end. Currently a lot of natural or synthetic surfactants have been on the market (for example, natural surfactant derived from animal or plant and synthetic surfactant -cationic, anionic or nonionic).

However, the selection of appropriate surfactants to prepare the stable tacrolimus injection is deemed difficult, for each of surfactant has different physical values such as hydrophile-lipophile balance (HLB) and critical micelle concentration (CMC), with diverse features in the application field.

The conventional technologies involving the solution formulations have confined their application scopes to some surfactants including polyoxyethylene hydrogenated castor oil.

To overcome the aforementioned shortcomings of the conventional injections, the inventors of this invention have carried out a lot of investigations to discover some surfactants with less toxicity and better stability.

Consequently, the inventors have invented injectable formulation of tacrolimus, characterized in that the use of macrogol 15 hydroxystearate contributes to settlement of the drawbacks the prior art has faced.

### DISCLOSURE OF INVENTION

### Technical Problem

The object of this invention is to provide tacrolimus injection that can be administered intravenously to human patients, without precipitation of the drug when diluted with saline or dextrose solution.

Another object of this invention is to provide tacrolimus injection that may optimize its stability without decrease of drug content during long-term storage, despite the inclusion of water-insoluble tacrolimus as an active ingredient.

### Technical Solution

To achieve the above objective, this invention is to provide an injectable formulation comprising tacrolimus as an active ingredient, macrogol 15 hydroxystearate as a surfactant and a non-aqueous solvent.

Tacrolimus injection has to safe with low toxicity, because it should be administered intravenously to human patients. Also, it must stable pharmaceutically without decrease of drug content or precipitation of the drug during long-term storage, or precipitation when diluted with saline or dextrose solution.

Thus, this invention is characterized by tacrolimus injection containing macrogol 15 hydroxystearate so as to minimize the undesirable properties.

This invention is described in more detail as set forth hereunder.

Tacrolimus injection of this invention is prepared such that tacrolimus and macrogol 15 hydroxystearate are dissolved in a non-aqueous solvent.

If tacrolimus only dissolves in the non-aqueous solvent, it maybe happens that precipitation of the drug during long-term storage, decrease of drug content, and precipitation of the drug when diluted with saline or dextrose solution.

Thus, this invention is characterized by another addition of macrgol 15 hydroxystearate to tacrolimus injection.

Macrogol 15 hydroxystearate is a nonionic surfactant with better chemical stability and less toxicity, the surfactant is well dissolved in water, ethanol and 2-propanol. Further, it can be used as a solubilizer of injectable formulations.

Tacrolimus injection of this invention containing macrgol 15 hydroxystearate is advantageous in that (1) the drug is not precipitated during long-term storage, (2) no decrease of the drug content ensures better stability, (3) the drug is not precipitated when diluted with saline or dextrose solution prior to administration.

It is preferred that the weight part of macrogol 15 hydroxystearate to tacrolimus is 20-100:1.

Tacrolimus injection of this invention is prepared such that tacrolimus and macrogol 15 hydroxystearate are dissolved in an appropriate non-aqueous solvent.

Any types of the pharmaceutically acceptable non-aqueous solvent may be used for this invention, but the solvent should be able to inject intravenously in dilution with saline or dextrose solution. The preferred non-aqueous solvent is ethanol anhydrous.

According to this invention, the non-aqueous solvent may be used in a clinically acceptable amount to dissolve both tacrolimus and macrgol 15 hydroxystearate sufficiently.

It is preferred that tacrolimus injection of this invention is intravenously administered to human patients in a form diluted with saline or dextrose solution. If deemed necessary, tacrolimus injection may contain pharmaceutically acceptable additive(s).

As the injection of this invention, tacrolimus is not precipitated, and the content of drug is not decreased significantly, during long-term storage.

And there is no precipitation when diluted with saline or dextrose solution, and it could be safely administered to human patients with less toxicity.

### Advantageous Effects

Tacrolimus injection of this invention is stable pharmaceutically during long-term storage, and it can be administered intravenously to human patients without precipitation of the drug when diluted with saline or dextrose solution.

Tacrolimus injection of this invention may significantly avoid a loss of the drug content during long-term storage.

### Best Mode for Carrying Out the Invention

This invention will now be described by reference to the following Examples. But these examples are not to be construed as a limitation of the scope of this invention which is defined in particular by the Claims.

### COMPARATIVE EXAMPLE 1

### Tacrolimus Injection using Polyoxyethylene Hydrogenated Castor Oil

In 1 ml ethanol anhydrous were dissolved to tacrolimus 5 mg and polyoxyethylene hydrogenated castor oil 60 (HCO-60) 200 mg, followed by filtration using a filter of 0.2 [symbol] , and an injectable form was prepared.

### EXAMPLE 1

### Tacrolimus Injection using Macrogol 15 Hydroxystearate

In 1 ml ethanol anhydrous were dissolved to tacrolimus 5 mg and macrogol 15 hydroxystearate (Solutol® HS 15, BASF Co.) 100 mg, followed by filtration using a filter of 0.2 [symbol] , and an injectable form was prepared.

### EXAMPLE 2

### Tacrolimus Injection using Macrogol 15 Hydroxystearate

In 1 ml ethanol anhydrous were dissolved to tacrolimus 5 mg and macrogol 15 Hydroxystearate (Solutol® HS 15, BASF Co.) 200 mg, followed by filtration using a filter of 0.2 [symbol] , and an injectable form was prepared.

### EXAMPLE 3

### Tacrolimus Injection using Macrogol 15 Hydroxystearate

In 1 ml ethanol anhydrous were dissolved to tacrolimus 5 mg and macrogol 15 Hydroxystearate (Solutol® HS 15, BASF Co.) 300 mg, followed by filtration using a filter of 0.2 [symbol] , and an injectable form was prepared.

### EXAMPLE 4

### Tacrolimus Injection using Macrogol 15 Hydroxystearate

In 1 ml ethanol anhydrous were dissolved to tacrolimus 5 mg and macrogol 15 hydroxystearate (Solutol® HS 15, BASF Co.) 400 mg, followed by filtration using a filter of 0.2 [symbol] , and an injectable form was prepared.

### EXAMPLE 5

### Tacrolimus Injection using Macrogol 15 Hydroxystearate

In 1 ml ethanol anhydrous were dissolved to tacrolimus 5 mg and macrogol 15 hydroxystearate (Solutol® HS 15, BASF Co.) 500 mg, followed by filtration using a filter of 0.2 [symbol] , and an injectable form was prepared.

### EXPERIMENTAL EXAMPLE 1

Each of tacrolimus injections, so prepared by comparative example 1 and Examples 1-5, was left at room temperature to confirm the crystallization, as shown in Table 1.

TABLE 1
Crystallization from tacrolimus Injections

| | Dilution | | | | | | |
|---|---|---|---|---|---|---|---|
| Tacrolimus | ratio by | Period until tacrolimus inj. become turbid (day) | | | | | |
| conc. | saline | Comparative | | | | | |
| (mg/ml) | solution | example 1 | Example 1 | Example 2 | Example 3 | Example 4 | Example 5 |
| 5 | 100 | ≧7 | ≧5 | ≧5 | ≧7 | ≧7 | ≧7 |
| | 1 | ≧7 | ≧7 | ≧7 | ≧7 | ≧7 | ≧7 |

Table 1 showed that no precipitation was observed from each of tacrolimus injections, so prepared by Comparative example 1 and Examples 1-5, when they were left at room temperature for more than 7 days.

Further, in a 100-fold dilution test of each of injections by saline solution, no change was observed from injections, so prepared by Comparative example 1 and Examples 3-5, when they were left at room temperature for more than 7 days. On the other hand, a slight white precipitation was found from some injections, so prepared by Examples 1-2, when they were left at room temperature for 5 days.

Therefore, it was noted that tacrolimus injection of this invention has better stability as no precipitation was observed during long-term storage conditions and/or by dilution in saline solution.

### EXPERIMENTAL EXAMPLE 2

Each of tacrolimus injections, so prepared by Comparative example 1 and Examples 1-5, was analyzed by HPLC to confirm the drug content (%) of tacrolimus Injections, as shown in Table 2.

TABLE 2
Drug Content of tacrolimus injection

Drug content of tacrolimus (%)

| Storage conditions | | Comparative example 1 | Example 3 |
|---|---|---|---|
| Initial period | | 100.0 | 100.0 |
| 40° C. | 1 month | 96.6 | 98.4 |
| | 6 months | 96.5 | 98.2 |
| | 12 months | 84.7 | 90.5 |
| 80° C. | 1 day | 95.2 | 96.4 |
| | 3 days | 90.8 | 93.3 |
| | 7 days | 81.9 | 85.2 |

Table 2 showed that the drug content of tacrolimus injection in all test conditions, so prepared by Example 3, was higher than that of tacrolimus injection by Comparative example 1.

Therefore, tacrolimus injection of this invention is more stable than conventional product, and there is no radical decrease of drug content during long-term storage.

1. An injectable preparation comprising tacrolimus, macrogol 15 hydroxystearate and ethanol anhydrous.

2. The injectable preparation according to claim 1, wherein the weight part of macrogol 15 hydroxystearate to tacrolimus is 20-100:1.
